(19) United States
(12) Patent Application Publication
(10) Pub. No.: US 2022/0370551 A1
(43) Pub. Date: Nov. 24, 2022

(54) Use of HDL in the prophylaxis of graft-versus-host disease

(71) Applicants: Etablissement Français du Sang, LA PLAINE SAINT DENIS (FR); INSTITUT NATIONAL DE LA SANTE ET DE LA RECHERCHE MEDICALE (INSERM), PARIS (FR); CENTRE HOSPITALIER REGIONAL UNIVERSITAIRE DE BESANCON, BESANCON (FR); UNIVERSITE DE FRANCHE-COMTE, BESANCON (FR)

(72) Inventors: Cécile CHAGUÉ, BESANCON (FR); Etienne DAGUINDAU, SERRE LES SAPINS (FR); Philippe SAAS, BESANCON (FR)

(21) Appl. No.: 17/641,472

(22) PCT Filed: Sep. 10, 2020
(86) PCT No.: PCT/EP2020/075306

§ 371 Date: Mar. 9, 2022

(30) Foreign Application Priority Data
Sep. 12, 2019 (FR) FR19 10075

(51) Int. Cl. A61K 38/17; A61P 37/06

(52) U.S. Cl. CPC A61K 38/1709; A61P 37/06

(57) ABSTRACT

This invention concerns a high-density lipoprotein (HDL) or HDL mimetic for use in the prevention and/or treatment of graft-versus-host disease (GvHD) or cytokine release syndrome in a subject.

This invention concerns the prevention and/or treatment of graft-versus-host disease.

The curative efficacy of bone marrow transplantation (BMT) is reduced considerably by graft-versus-host disease (GvHD), which is associated with significant morbidity and mortality.

GvHD is a pathology for which corticosteroid therapy remains the first-line treatment. However, corticosteroid therapy is, on the one hand, associated with numerous iatrogenic complications (diabetes, disorders of osseous metabolism with a risk of aseptic osseous necrosis, arterial hypertension, dyslipidaemia, etc.), whilst, on the other, the prognosis is poor in the event of resistance to this treatment. There is no consensus regarding treatment in these severe cases, and immunosuppressive approaches that interfere with the cell cycle of T lymphocytes remain the only tools available.

There is thus a significant need for alternative treatments for GvHD that avoid such side effects.

This invention seeks to meet this need.

Donor T cell subgroups are recognised to be the main mediators and effectors of acute GvHD. Interactions between T lymphocytes and antigen-presenting cells (APCs) from the donor and the host are required in order to achieve the status of ‘activated alloreactive T cell’ that generates the cytotoxic attack against target organs. However, the preliminary activation of APCs by exogenous or endogenous alarm signals from damaged or ‘distressed’ cells is critical in order to effectively recruit and activate T lymphocytes. Exogenous signals are a group of very widespread natural microbial patterns known as ‘pathogen-associated molecular patterns’ (PAMP), and it is believed that they are translocated from the microbiota when the physical barriers of the body, in particular the intestinal mucosa, are weak. Of these PAMPs, lipopolysaccharide (LPS) is the one most heavily studied in relation to the pathophysiology of GvHD.

The inventors identified an increase in free active plasma LPS after an allograft, and then explored its metabolism in order to identify the critical stage that was altered during allogeneic bone marrow transplantation. In healthy individuals, LPS is generally transported to the liver by HDLs, where it is then eliminated via the bile. Various plasma transporters are implicated in the loading of LPS onto HDLs. Here, the inventors have shown that the limiting factors that explains the increase in free active LPS was a drop in circulating HDL.

This invention arises from the inventors' unexpected discovery that the repeated administration of HDL lipoproteins neutralises LPSs in the setting of GvHD, reducing the maturation of APCs and the activation of T Tc1 lymphocytes, the effector cells of GvHD, and thus reducing the occurrence and severity of acute GvHD.

Thus, for the first time, the treatment proposed here by the inventors—HDL administration—allows for prevention and treatment of GvHD, relying not on toxic immunosuppressive treatments, but on the modulation of lipid metabolism, which has no impact on the quality of post-transplant immune reconstitution and for which the expected toxicity for other organs is minimal.

This is particularly true in the liver, where the inventors have shown reduced CD8+T-cell infiltration, as well as limited activation of resident and non-resident macrophages following treatment with HDL in an experimental model of GvHD.

Cooke et al. (2001) J. Clin. Invest. 107:1581 studied the use of a competitive LPS inhibitor, compound B975, in a murine bone marrow transplant model. Unlike compound B975, HDL is not a competitive LPS inhibitor; rather, it neuralises and eliminates LPS.

Moreover, HDLs have an anti-inflammatory effect independent of LPS, which makes them even more interesting for the prevention and/or treatment of GvHD, the pathophysiology of which is characterised by a significant inflammatory component.

Thus, this invention concerns a high-density lipoprotein (HDL) or HDL mimetic for use in the prevention and/or treatment of graft-versus-host disease (GvHD) or cytokine release syndrome in a subject.

### DETAILED DESCRIPTION OF THE INVENTION

### HDL and HDL Mimetics

‘High-density lipoprotein (HDL)’ refers here to the smallest, densest group of lipoprotein particles.

HDLs are well known to persons skilled in the art, and are typically lipoproteins rich in cholesterol and phospholipids that comprise the apolipoproteins A1, A-II, A-IV, C-I, C-II, C-III, and E, which have a density of between 1.063 and 1.210 and a diameter ranging between 5 and 12 nm.

Typically, the HDLs used in the context of the invention are isolated from the blood of healthy donors, in particular from healthy donor plasma, e.g. by ultracentrifugation-based separation techniques.

Alternatively, the HDLs used in the invention are reconstituted from purified or recombinant apolipoprotein Al, as defined infra, and from selected lipids, in particular soya phospholipids.

Thus, examples of HDLs that may be used in the invention include CSL111, as described in Tardif et al. (2007) JAMA 297:1675-1682, CSL112, as described in Diditechenko et al. (2013) Arterioscler. Thromb. Vasc. Biol. 33:2202-211, CER-001, as described in Tardif et al. (2014) Eur. Heart J. 35:3277-3286, ETC-216 (also known as MDCO-216, an HDL reconstituted from ApoA1_{Milano}).

‘HDL mimetic’ refers here to molecules that mimic the function of HDL.

Examples of HDL mimetics include gold nanoparticles covered in phospholipids and ApoA1 or ApoA1 mimetics, and HDLs reconstituted from ApoA1 mimetics such as the molecule ETC-642 described in Di Bartolo et al. (2011) Lipids Health 10:224.

### Medical Applications

This invention concerns a high-density lipoprotein (HDL) as defined in the section HDL and HDL Mimetics supra or an HDL mimetic as defined in the section HDL and HDL Mimetics supra, for use in the prevention and/or treatment of graft-versus-host disease (GvHD) or cytokine release syndrome in a subject.

This invention also concerns the use of a high-density lipoprotein (HDL) as defined in the section HDL and HDL Mimetics supra or an HDL mimetic as defined in the section HDL and HDL Mimetics supra, for the production of a medicament for the prevention and/or treatment of graft-versus-host disease (GvHD) or cytokine release syndrome in a subject.

The invention further concerns a method for prevention and/or treatment of GvHD or cytokine release syndrome, comprising administering a therapeutically effective amount of an HDL as defined in the section HDL and HDL Mimetics supra or an HDL mimetic as defined in the section HDL and HDL Mimetics supra in a subject in need thereof.

‘Treatment’, ‘treating’, etc. refer here to attaining one or more of the following results, in whole or in part: partial or total reduction of the extent of the disease, improvement of a clinical symptom or indicator associated with the disease, inhibiting or preventing progression of the disease, or totally or partially delaying, inhibiting, or preventing the occurrence of a relapse of the disease.

In the context of the invention, ‘prevention’, ‘preventing’, etc. refers to any index for the success of protecting a subject or patient (e.g. a subject or patient at risk of developing a disease) from developing, contracting, or having a disease, including the prevention of one or more symptoms of the disease.

‘Subject’ refers to a mammal, preferably a human. Preferably, the subject treated in the context of the invention has a cancer, in particular a haematological malignancy (e.g. leukaemia, lymphoma, or myeloma), or a non-malignant haematological disorder such as primary immunodeficiency, medullar aplasia, or myelodisplasia.

‘Graft-versus-host-disease’, ‘GvHD’, ‘GvH’, or ‘graft-versus host reaction’ refers to any T-cell-mediated immune response in which donor lymphocytes react with host antigens, typically following a bone marrow graft or bone marrow allotransplant.

GvHD is described as ‘acute’ or ‘chronic’ depending on its time to first occurrence and, above all, the type of clinical manifestations respectively including signs of inflammation or fibrosis. One form, known as ‘overlap syndrome’, refers to GvHD that includes characteristics of acute and chronic GvHD. Acute GvHD may vary in severity between mild and very severe. The severity of chronic GvHD, on the other hand, is usually defined as ‘limited’ or ‘extensive’.

Acute GvHD mostly occurs within the first 100 days following an allotransplant. It commonly affects the skin, the liver, and the intestines, but it may also affect other organs. Acute GvHD maybe classified according to the severity of its symptoms:
grade 1: mild symptoms
grade 2: moderate symptoms,
grade 3: severe symptoms,
grade 4: very severe symptoms.

The symptoms of acute GvHD typically include: Burning sensation and redness of the skin of the palms of the hands or the soles of the feet, skin rash that may spread over the entire body, blisters and desquamation, anorexia, nausea, vomiting, abdominal pain, malabsorption diarrhoea, hepatic cytolysis associated with jaundice, liver abnormalities up to and including hepatocellular failure.

Classically, chronic GvHD occurs starting in the third month following the allotransplant. It may last several months or the patient's lifetime. Chronic GvHD may occur immediately after acute GvHD or a symptom-free period. The symptoms of chronic GvHD typically include: Skin disorders such as dryness, skin rash, itching, desquamation, poikilodermia, loss of skin elasticity up to including more or less extensive scleroderma. Mucosal and/or eye dryness is commonly present, and results in a sensation of burning/foreign body in the eyes, xerostomia (dry mouth), which may or may not be associated with oral lichen and/or ulceration. In the digestive context, chronic GvHD manifests as anorexia, abdominal pain, diarrhoea, malabsorption, nausea/vomiting. Numerous other organs may be affected, e.g. the lungs (pulmonary fibrosis) or the musculoskeletal system (polyarthritis, cramps). It should be noted that chronic extensive GvHD is frequently associated with immune reconstitution failure, which exposes the patient to opportunistic infectious complications.

In one particular embodiment, the GvHD prevented or treated in the context of the invention is acute GvHD.

In one particularly preferred embodiment, the GvHD prevented or treated in the context of the invention is acute hepatic GvHD.

As noted supra, GvHD typically occurs following a bone marrow transplant.

Thus, in one particular embodiment, to prevent or treat GvHD, in particular acute GvHD, the subject being treated has undergone/is to undergo a transplant, in particular an allotransplant, of bone marrow.

More specifically, in one particular embodiment that seeks to prevent GvHD, the subject being treated is to undergo a transplant, in particular an allotransplant, of bone marrow.

In an alternative embodiment that seeks to treat GvHD, the subject to be treated has undergone a transplant, in particular an allotransplant, or bone marrow.

In a particular embodiment, the subject being treated has a disorder of digestive permeability, which may typically be due to a treatment prior to the transplant, such as chemotherapy and/or radiotherapy. Without wishing to be bound by theory, these treatments may affect the integrity of the intestinal barrier, inducing apoptosis in intestinal epithelial cells, promoting immune cell infiltration, and disrupting crypt/villi structures.

‘Cytokine release syndrome (CRS)’ refers here to a systemic inflammatory response that may be triggered by various factors, such as infections and certain medications. Thus, CRS is typically observed as a side effect following the administration of antibody therapies, non-protein anti-cancer therapies, the administration of CAR-T lymphocytes, as well as bone marrow allotransplantation or GvHD. This syndrome is typically characterised by severe dyspnoea with bronchospasm and hypoxia, associated with fever, chills, tremor, urticaria, and angio-oedema, and may result in acute respiratory failure and death.

Thus, in a particular embodiment that seeks to prevent and/or treat CRS, the subject being treated has been treated/is being treated with anti-cancer agents, in particular anti-cancer antibodies, or with CAR-T cells, or has undergone/is to undergo a bone marrow transplant.

In a preferred embodiment that seeks to prevent and/or treat CRT, the subject being treated is being treated/has been treated with CAR-T cells.

The HDL, as defined in the section HDL and HDL Mimetics supra, or the HDL mimetic, as defined in the section HDL and HDL Mimetics supra, is typically administered to the subject in need thereof in a therapeutically effective amount.

A ‘therapeutically effective amount’ refers here to an amount of active ingredient that is sufficient to eradicate, modify, control, or eliminate the disease. A ‘therapeutically effective amount’ also refers to an amount of active ingredient that delays or minimizes the extent of the disease. It also refers to the amount of active ingredient that provides a therapeutic benefit in the treatment or management of the disease. Lastly, ‘therapeutically effective amount’ refers to an amount of active ingredient, alone or in combination with other therapies, that provides a therapeutic benefit in the treatment or management of the disease, including an improvement in the symptoms associated with the disease.

The amount that will be therapeutically effective depends, of course, on the active ingredient in question, the mode of administration, the therapeutic indication, and the age and health of the patient.

Advantageously, the active ingredient used in the invention is in the form of a pharmaceutical composition, optionally further comprising a pharmaceutically acceptable excipient.

‘Pharmaceutically acceptable’ refers here to compositions and molecular entities that do not cause side effects, allergic reactions, or other adverse effects when administered to a subject. Thus, a pharmaceutically acceptable excipient or vehicle is an encapsulating agent, a diluent, a support, or any other non-toxic liquid, semi-solid, or solid formulation agent.

Pharmaceutical compositions used in the invention are typically prepared so as to adapt them to the mode of administration. Acceptable pharmaceutical excipients are typically determined in part by the composition being administered, as well as the particular technique used to administer the composition.

The dosage of the compounds to be administered depends on the individual case, and, as is well known to persons skilled in the art, must be adapted to the individual circumstances in order to obtain a therapeutically effective amount and an optimum effect. The dosage that will be therapeutically effective is specific to each patient, and will depend, in particular, on various factors, including the disorder being treated and the severity thereof, the activity of the specific compound used, the specific composition used, the age, weight, general health, gender, and diet of the patient, the time of administration, route of administration, and the excretion rate of the specific compound used, the duration of treatment, the drugs used in combination with the specific compound used, and similar factors that are well known in the medical field. For example, it is well known to persons skilled in the art to start with lower dosage levels of a compound than are required in order to attain the desired therapeutic effect and to progressively increase the dosage until that desired effect is attained.

The daily dose may be administered in a single dose, or, in particular when larger amounts are administered, it may be divided into several individual doses.

The dosage of the active ingredient depends particularly on the mode of administration, and can be easily determined by persons skilled in the art. A therapeutically effective amount (unit dose) of a compound may be between 0.01 and 500 mg/kg, preferably between 0.1 and 500 mg/kg, more preferably between 0.1 and 250 mg/kg, more preferably between 0.1 and 100 mg/kg, more preferably between 0.1 and 50 mg/kg, even more preferably between 1 and 20 mg/kg, in one or more weekly administrations over several weeks or months. The effective unit dose may thus be easily determined from a dose calculated for an ‘average’ patient weighing 70 kg.

Typically, the HDL, as defined in the section HDL and HDL Mimetics supra, or the HDL mimetic, as defined in the section HDL and HDL Mimetics supra, is administered to the subject in need thereof in a dose of 20 mg/kg.

In a particular embodiment, the HDL, as defined in the section HDL and HDL Mimetics supra, or the HDL mimetic, as defined in the section HDL and HDL Mimetics supra, is administered repeatedly, preferably every day, to the subject in need thereof.

In a particular embodiment, when the subject being treated is to undergo a bone marrow transplant, the HDL, as defined in the section HDL and HDL Mimetics supra, or HDL mimetic, as defined in the section HDL and HDL Mimetics supra, is administered prior to the BMT, preferably during the conditioning phase, typically 7 days before the BMT.

Preferably, the HDL as defined in the section HDL and HDL Mimetics supra, or HDL mimetic, as defined in the section HDL and HDL Mimetics supra, is administered prior to the BMT as defined supra, and is administered again after the BMT, typically 1-3 days after the BMT, preferably 1 day after the BMT, more preferably repeatedly between 1 and 24 days after the BMT.

Alternatively, the administration of the HDL, as defined in the section HDL and HDL Mimetics supra, or HDL mimetic, as defined in the section HDL and HDL Mimetics supra, begins after the BMT, typically 1-7 days after the BMT, typically 1 day after the BMT, preferably repeatedly between 1 and 24 days after the BMT.

The compositions used in the invention may be solid, liquid, or semi-solid, adapted to various routes of administration (oral, rectal, nasal, intraocular, local (e.g. topical, transdermal, buccal, vaginal, or sublingual) or parenteral (e.g. subcutaneous, intramuscular, intravenous, or intradermal)).

Preferably, the HDL or HDL mimetic is administered intravenously.

Intravenous formulations contain the active ingredient, which is dissolved, suspended, or emulsified in a sterile vehicle, optionally in the presence of emulsifiers, stabilisers, buffering agents, and other conventional additives; they are normally distributed in flasks or bottles, and maybe stormed in dry form for reconstitution with water or an appropriate vehicle prior to use.

Solid pharmaceutical compositions may be tablets, capsules, powders, granules, pills, powders for reconstitution, etc. They may contain excipients such as binding agents, bulking agents, diluents, compacting agents, lubricants, detergents, colouring agents, flavourings, and wetting agents. The tablets may be coated by methods well known in the art. Suitable bulking agents include cellulose, mannitol, lactose, and the like.

Liquid compositions for oral administration may be in the form of aqueous or oily suspensions, solutions, emulsions, syrups, or elixirs, or they may be in the dry form for reconstitution with water or a suitable vehicle prior to use. They may contain conventional additives, e.g. suspending agents such as sorbitol, syrup, methylcellulose, gelatin, hydroxyethylcellulose, carboxymethylcellulose, aluminium stearate gel, or hydrogenated edible fats, emulsifiers such as lecithin, sorbitan monooleate, or acacia gum; non-aqueous carriers (which may comprise edible oils) such as almond oil, walnut oil, or fractionated coconut oil, oily esters such as glycerin, propylene glycol, or ethyl alcohol esters; preservatives such as methyl or propyl p-hydroxybenzoate or sorbic acid, or, optionally, conventional flavouring or colouring agents.

In this application, the term ‘comprise’ shall be construed to cover all of the characteristics specifically mentioned, and, optionally, as certain additional characteristics that are not specified. Moreover, the use of the term ‘comprising’ also describes the embodiment in which no characteristics are present other than those specifically mentioned (i.e. ‘consisting of’).

This invention will be described in greater detail in the following figures and examples.

### EXAMPLES

### Example 1

In this example, the inventors will show that the intravenous administration of allogeneic T lymphocytes induces premature translocation of LPS as well as a low degree of neutralisation of its inflammatory properties. This LPS is mostly found in free form in the plasma following BMT.

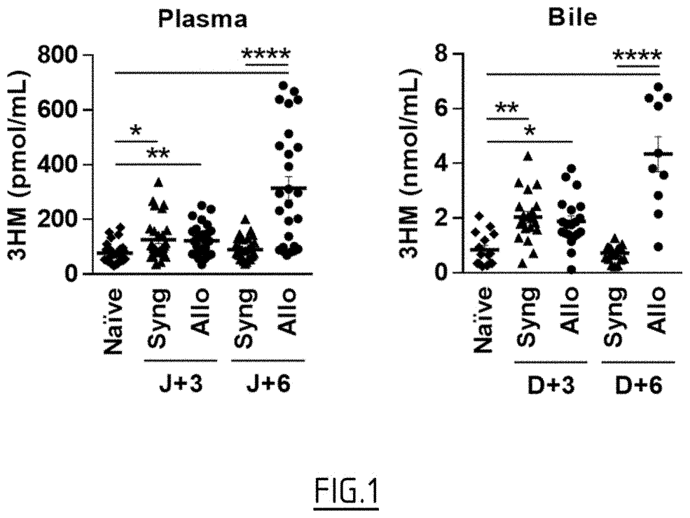

In an established murine model for GvHD, consisting of lethally (8.5 Gy) irradiated BALB/c recipient mice implanted with 5×10⁶ T-lymphocyte-depleted bone marrow and 1×10⁶ splenic T lymphocytes from BALB/c (syngeneic, syng) or C57Bl/6 (allogeneic, allo) donors, 3-hydroxymyristic acid (3HM)—the most common hydroxylated fatty acid found in the A lipid of LPS—was quantified in the plasma and bile of the recipient mice 3 and 6 d post BMT using the Endoquant® technique based on HPLC coupled with tandem mass spectrometry (Plasma: n=26-32 mice/group of 6 independent experiments, Kruskal-Wallis and Dunn post-test, *: p<0.05, **: p<0.01, ****: p<0-0001: Bile: n=26-21 mice/group of 3 independent experiments, post unidirectional ANOVA and Bonferroni test, *: p<0.05, **: p<0.01, ****: p<0.0001) (FIG. 1).

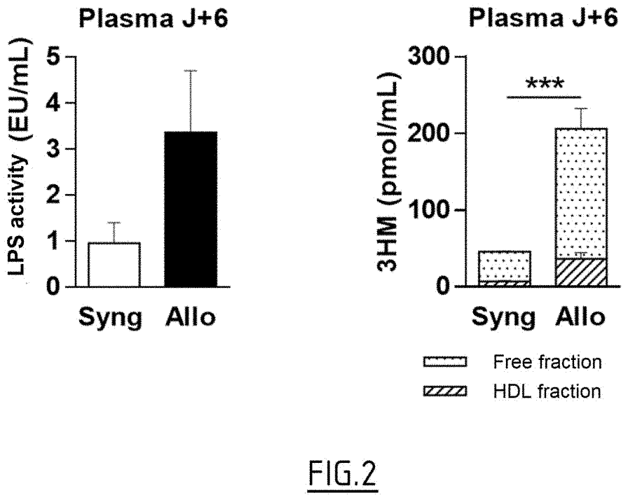

The inventors found that this increase in plasma LPS following bone marrow allotransplantation is principally LPS in its free form. The inventors confirmed the increase in LPS post allotransplant using the LAL test (Limulus Amoebocyte Lysate, based on the coagulation of the lysate of amoebocytes contained in limulus blood only in the presence of active, i.e. free, LPS) (n=9 mice/group in 3 independent experiments) (FIG. 2, left). This was confirmed by separating the free form of LPS 8not associated with circulating lipoproteins) and the HDL-associated form by ultracentrifugation, followed by quantification by the Endoquant® technique (n=3-4 mice/group in 1 experiment, bidirectional ANOVA post-test and Bonferroni test, ***: p<0.001) (FIG. 2, right).

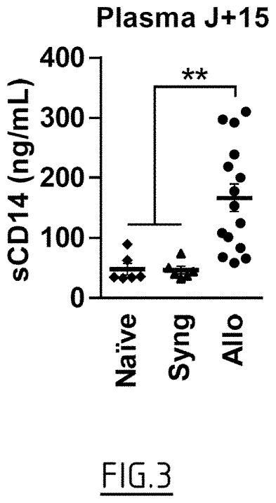

The inventors found that the soluble form of CD14 (sCD14) was increased in the plasma of allorecipient mice at d+15 post transplantation (n=6-15 mice/group in 3 independent experiments, Kruskal-Wallis post-test and Dunn test, **: p<0.01) (FIG. 3).

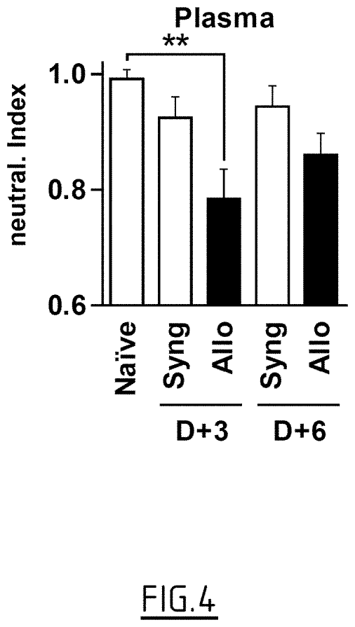

The ability of the plasma of the recipient mice to neutralise the activity of a known concentration of LPS was studied in vitro. In brief, HEK-Blue TLR4/CD14/MD-2 reporter cells were cultured in the presence of 0.5% plasma and 0.01 EU/ml standard LPS. The inventors showed that, d+3 post transplantation, the plasma of the allorecipient mice had a neutralising effect on the commercial PS added at a known concentration (0.01 EU/mL, Invivogen) that was reduced by 21.6%, compared to the naïve mice. At d+6, the decrease was equal to 14% (n=11-16 mice/group of 2 independent experiments, post unidirectional ANOVA and Bonferroni test, **: p<0.01) (FIG. 4).

### Example 2

In this example, the inventors will show that the reduced neutralisation capability appears to be due to altered reverse transport of LPS in allorecipient mice.

Reverse LPS transport (RLT) is a metabolic pathway analogous to reverse cholesterol transport, in which lipophilic substances are transported to the liver by lipoproteins (mainly HDL) in order to be recycled or eliminated in the bile.

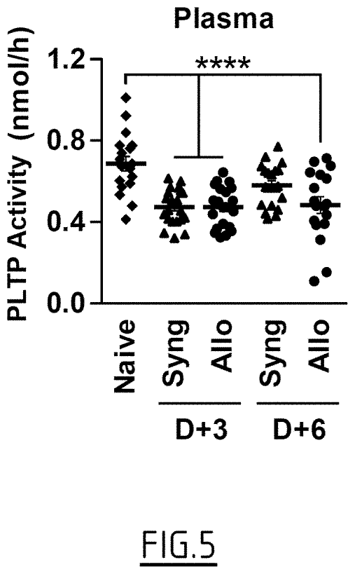

PLTP (Phospholipid transfer protein) plays an important role in RLT, and is capable of loading LPS onto HDLs. The inventors have shown that its activity, as measured by a commercially available kit, was slightly reduced by full body irradiation (FBI) (d+3 post transplantation) and the administration of allogeneic T lymphocytes (d+6) (n=18-21 mice/group in 3 independent experiments, post unidirectionalANOVA and Bonferroni test, ****: p<0.0001) (FIG. 5).

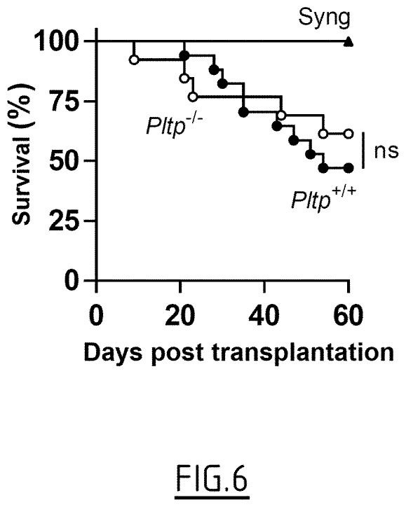

Nonetheless, the inventors showed that PLTP activity was not the main effector in the GvHD context, given that its total absence did not increase mortality in allotransplanted mice. In this model, lethally irradiated (10 Gy) Pltp+/+ or Pltp−/− C57Bl/6 mice were implanted with 20×10⁶ bone marrow and 5×10⁶ splentic T lymphocytes from C57Bl/6 (syngeneic, syng) or C3H (allogeneic, alto) donors (n=12-17 mice/group in 2 independent experiments, log-rank test, ns: insignificant, p=0.5338) (FIG. 6).

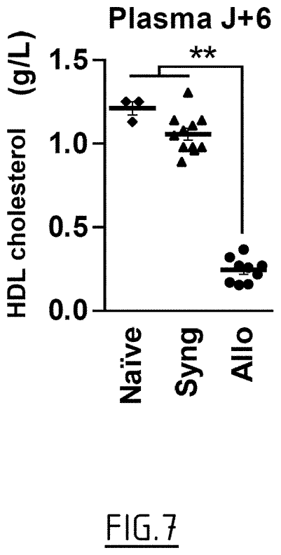

The inventors showed that the principal limiting factor appeared to be the available of LPS transporters, given that an early drop (d+6) in the plasma HDL concentration was observed in two murine GvHD models. FIG. 7 shows the results obtained with the C57Bl/6→BALB/c model (n=3-11 mice/group in 2 independent experiments, post Kruskal-Wallis and Dunn test, **: p<0.01).

### Example 3

In this example, the inventors will show that the loss in apolipoprotein A1 (ApoA1) synthesis and circulating HDL increases the severity of GvHD.

In order to study the dominant effect of HDL in RLT in the setting of GvHD, lethally irradiated (10 Gy) C57Bl/6 mice that either expressed (WT) or did not express (Apoa1^{tm1Unc}) the ApoA1 gene were implanted with 20×10⁶ bone marrow and 5×10⁶ splenic T lymphocytes from C57Bl/6 (syngeneic, syng) or C3H (allogeneic, allo) donors.

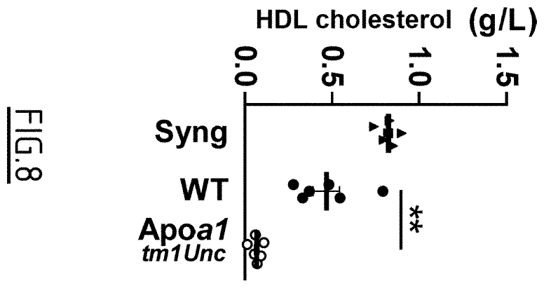

ApoA1 is the major apolipoprotein for the formation of HDL, and the inventors have shown that its absence aggravated the drop in HDL in ApoA1^{tm1Unc} recipient mice (d+6), resulting in near-total absence of circulating HDL in plasma (0.067 g/l) (n=6 mice/group in 2 independent experiments, Mann-Whitney test, **: p<0.01) (FIG. 8).

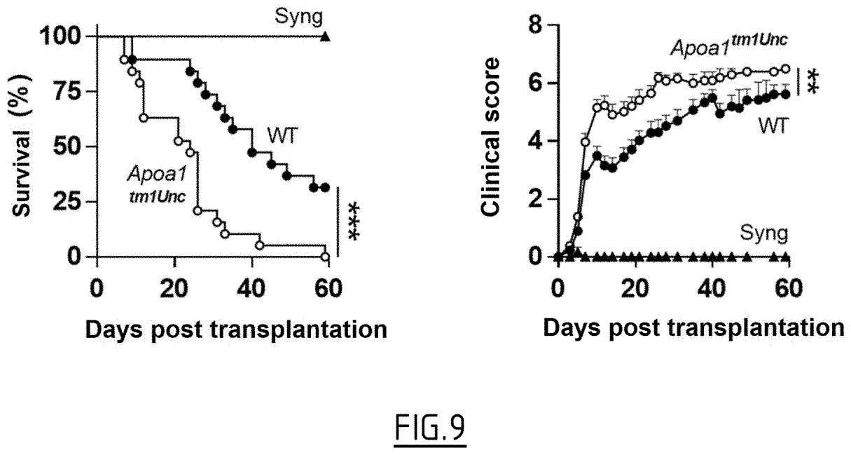

The inventors showed that the absence of HDL in allorecipient mice exacerbated mortality and severity of GvHD post transplantation (n=9-19 mice/group in 3 independent expeirments, log-rank survival test, ***: p<0.001 and Kruskal-Wallis and Dunn post-tests on ASC for clinical scoring, **: p<0.01) (FIG. 9).

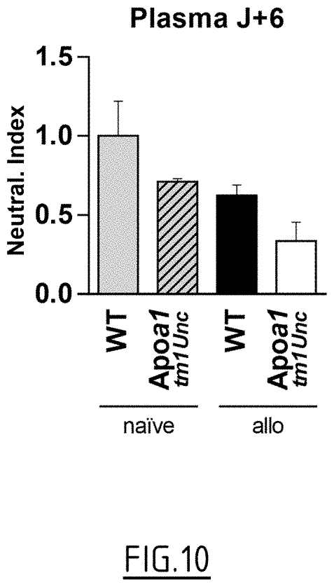

The inventors showed that the absence of HDL appeared to be associated with altered LPS neutralisation capability, as evaluated by the HEK-Blue TLR4/CD14/MD-2 reporter cell method (preliminary data, n=3 mice/group in 1 experiment) (FIG. 10).

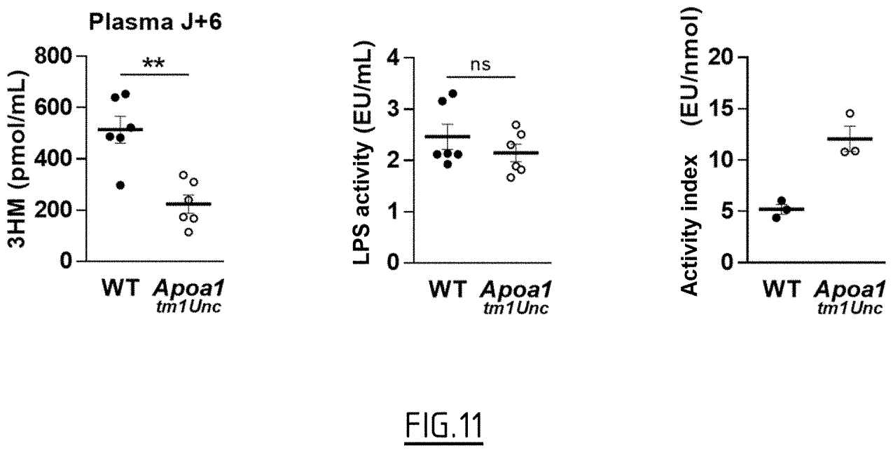

Lastly, the inventors showed that ApoA1^{tm1Unc} recipient mice had less ability to neutralise LPS d+6 post transplantation. Indeed, ApoA1^{tm1Unc} recipient mice have lower plasma 3HM concentrations (i.e. total LPS) than wild-type recipient mice (WT) (n=6 mice/group in 2 independent experiments, Mann-Whitney test, **: p<0.01) (FIG. 11, left). On the other hand, these 2 groups of recipient mice have the same amount of active LPS as measured by the LAL test (n=3 mice/group in 1 experiment) (FIG. 11, centre). This is reflected in a lower LPS neutralisation capability or activity index than in ApoA1^{tm1Unc} recipient mice (FIG. 11, right).

### Example 4

In this example, the inventors will show that the loss of synthesis of ApoA1 and HDL accelerates the maturation of dendritic cells and promotes IFN-γ production by T lymphocytes T in the spleen.

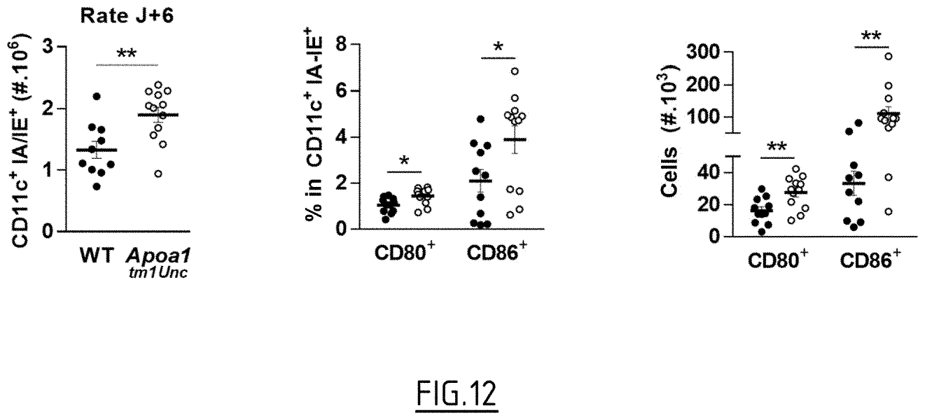

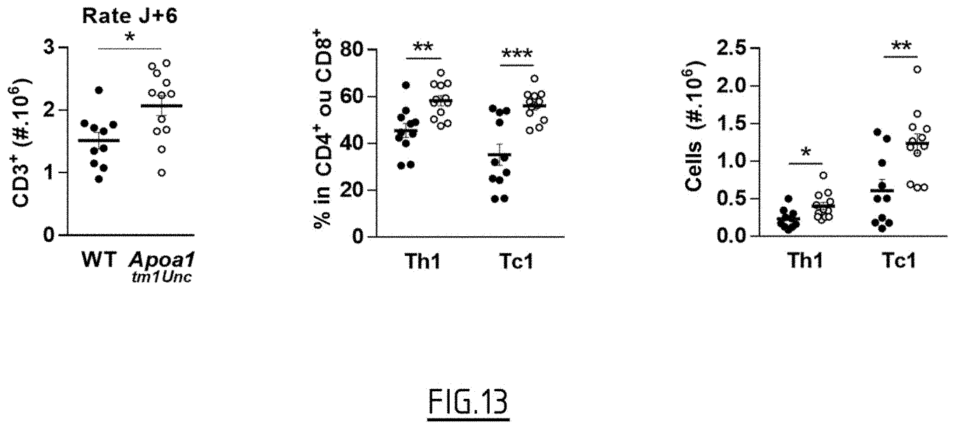

The maturation of conventional dendritic cells (DC) (FIG. 12) and the polarisation of T lymphocytes (FIG. 13) were evaluated by flow cytometry 6 days post transplantation at the level of the spleens of lethally irradiated (10 Gy) C57Bl/6 mice expressing (WT) or not expressing (Apoa1^{tm1Unc}) the ApoA1 gene and implanted with 20×10⁶ bone marrow and 5×10⁶ splenic T lymphocytes from C57Bl/6 (syngeneic, syng) or C3H (allogeneic, allo) donors.

The inventors showed that the absolute number of living DC CD3⁻CD19⁻ CD11c⁺IA−IE⁺ was significantly increased in the spleens of Apoa1^{tm1Unc} recipient mice (FIG. 12, left). These DCs show increased expression of maturation markers (CD80 and CD86) compared to the DCs of WT allotransplanted mice in terms of the percentage (FIG. 12, centre) and absolute number (FIG. 12, right) of positive cells (n=11-12 mice/group in 3 independent experiments, unpaired t test or Mann-Whitney test, *: p<0.05, **: p<0.01, ***: p<0.001).

The inventors have also shown that the absolute number of live splenic CD3⁺ T lymphocytes T was increased in Apoa1^{tm1Unc} recipients (FIG. 13, left). The proportions of cells expressing IFN-γ amongst CD3⁺CD4⁺ cells (Th1 lymphocytes) and CD3⁺CD8⁺ cells (Tc1 lymphocytes) were higher in Apoa1^{tm1Unc} recipients (FIG. 13, centre), and represented a greater absolute number of cells (FIG. 13, right) after 4 h of phorbol-myristate-acetate/ionomycin stimulation (n=11-12 mice/group in 3 independent experiments, unpaired t test or Mann-Whitney test, *: p<0.05, ** : p<0.01, ***: p<0.001).

### Example 5

In this example, the inventors will show that the loss of ApoA1 synthesis and circulating HDLs may increase T cell infiltration at the level of a target organ of GvHD, the Iver, and promotes IFN-γ production by these T cells that infiltrate the liver.

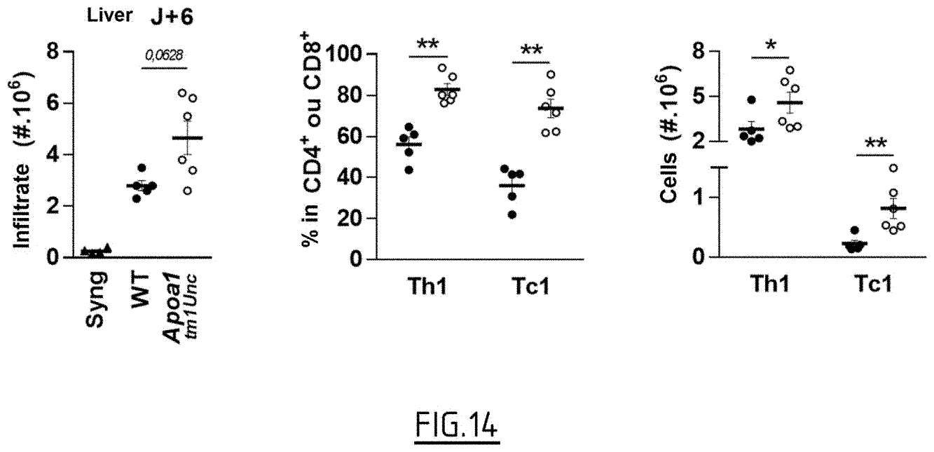

The polarisation of T lymphocytes (FIG. 14) isolated from the liver was evaluated by flow cytometry 6 days post transplantation of lethally irradiated (10 Gy) C57Bl/6 mice expressing (WT) or not expressing (Apoa1^{tm1Unc}) the ApoA1 gene and implanted with 20×10⁶ bone marrow and 5×10⁶ splenic T lymphocytes from C57Bl/6 (syngeneic, syng) or C3H (allogeneic, allo) donors.

The inventors showed that the absolute number of live CD3⁺ T cells present in the liver was slightly increased in Apoa1^{tm1Unc} recipients (n=5-6 mice/group in 1 experiment, Mann-Whitney test, p=0.0628) (FIG. 14, left). The proportions of cells expressing IFN-γ amongst CD3⁺CD4⁺ cells (Th1 lymphocytes) and CD3⁺CD8⁺ cells (Tc1 lymphocytes) were higher in Apoa1^{tm1Unc} recipients (FIG. 14, centre), and represented a greater absolute number of cells (FIG. 14, right) after 4 h of phorbol-myristate-acetate/ionomycin stimulation (n=5-6 mice/group in 1 experiment, Mann-Whitney test, *: p<0.05, **: p<0.01).

### Example 6

In this example, the inventors will show that the loss of ApoA1 synthesis and circulating HDLs results in an increase in hepatic macrophages, mainly non-resident macrophages derived from monocytes. These hepatic macrophages produce the proinflammatory cytokines TNF-α et IL-6 to an increased degree.

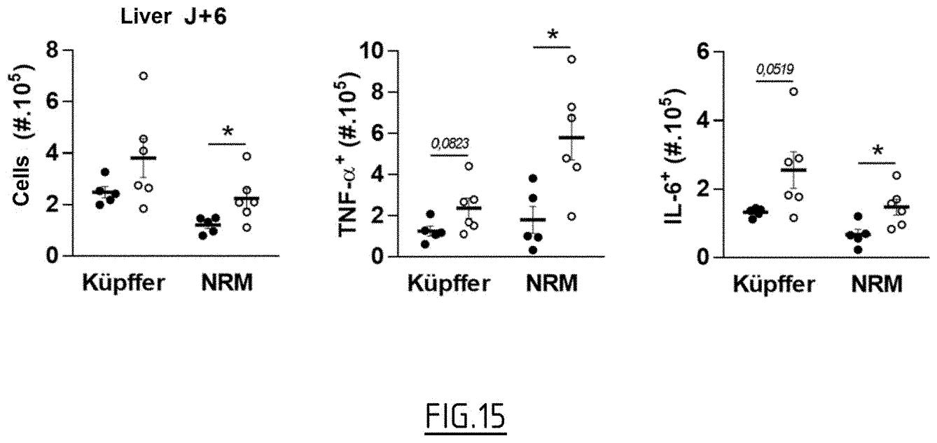

The production of cytokines by resident hepatic (Küpffer cells) and non-resident (NRM) macrophages (FIG. 15) was evaluated by flow cytometry 6 days post transplantation of lethally irradiated (10 Gy) C57Bl/6 mice expressing (WT) or not expressing (Apoa1^{tm1Unc}) the ApoA1 gene and implanted with 20×10⁶ bone marrow and 5×10⁶ splenic T lymphocytes from C57Bl/6 (syngeneic, syng) or C3H (allogeneic, allo) donors.

The inventors showed that the absolute number of resident (Küpffer cells) and non-resident (NRM) hepatic macrophages present in the liver was increased in Apoa1^{tm1Unc} recipients (n=5-6 mice/group in 1 experiment, Mann-Whitney test, *: p<0.05) (FIG. 15, left). The absolute number of cells expressing TNF-α (FIG. 15, centre) or IL-6 (FIG. 15, right) amongst F4/80^{int} CD11b^{high} cells (non-resident macrophages, NRM) was greater in Apoa1^{tm1Unc} recipients after 4 hours of LPS stimulation (n=5-6 mice/group in 1 experiment, Mann-Whitney test, *: p<0.05). With regard to F4/80^{high} CD11b^{high} cells (Küpffer cells) expressing these proinflammatory cytokines, there is an upward tendency (n=5-6 mice/group in 1 experiment, Mann-Whitney test, p=0.0823 and p=0.0519, for TNF-α and IL-6, respectively).

### Example 7

In this example, the inventors will show that the IV administration of HDL reduces the intensity of GvHD and neutralises the available LPS.

To test the value of prophylactic IV administration of HDL to moderate the severity of GvHD, lethally irradiated (8.5 Gy) BALB/c recipients implanted with 5×10⁶ T lymphocyte-depleted bone marrow and 1×10⁶ splentic T lymphocytes from BALB/c (syngeneic, syng) or C57Bl/6 (allogeneic, allo) donors were treated by IV administration of HDLs isolated from human plasma (20 mg/kg) 3 times per week between d−1 and d+24 post transplantation.

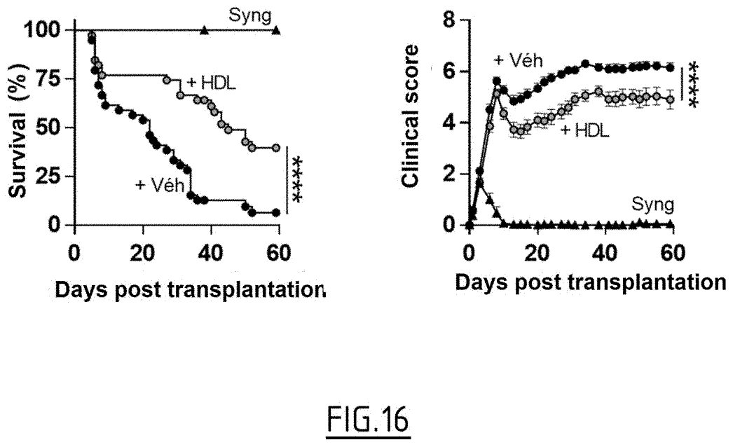

The inventors showed that IV HDL administration reduced the mortality (FIG. 16, left) and severity (FIG. 16, right) of GvHD in allotransplanted mice (n=19-39 mice/group in 4 independent experiments, log-rank survival test and unidirectional ANOVA post-test and Bonferroni on ASC for clinical scoring, ****: p<0.0001).

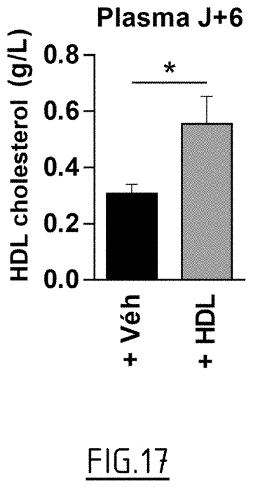

The inventors also showed that IV administration of isolated HDLs restored the circulating HDL level at d+6 (n=10-11 mice/group in 3 independent experiments, Mann-Whitney test, *: p<0.05) (FIG. 17).

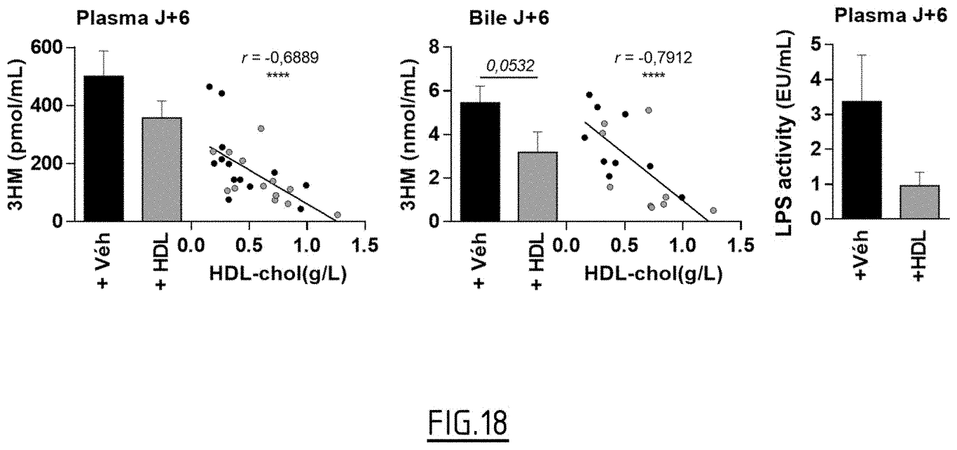

Lastly, the inventors showed that IV HDL administration appeared to reduce the 3ML level in the plasma and bile of allotransplanted mice (n=9-13 mice/group in 4 independent experiments, Mann-Whitney test). Plasma (FIG. 18, left) and biliary (FIG. 18, centre) 3HM levels correlated strongly with the circulating HDL level (n=13 mice/group in 4 independent experiments, respectively, Mann-Whitney test; n=26 pairs, non-parameterised Spearman correlation test, and n=9-10 mice/group in 4 independent experiments, Mann-Whitney test; n=19 pairs, non-parameterised Spearman correlation test, ****: p<0.0001). 3HM concentrations detected by the Endoquant® technique represent the total amount of LPS present in biological fluids. IV HDL administration also appears to reduce the endotoxic activity of circulating LPS as measured by LAL (n=7-9 mice/group in 3 independent experiments) (FIG. 18, right).

### Example 8

In this example, the inventors will show preliminary data showing that IV HDL administration may limit systemic inflammation associated with acute GvHD.

Preliminary data obtained by the inventors suggest that IV administration of HDL isolated from human plasma (20 mg/kg) 3 times per week may reduce the systemic inflammation caused by acute GvHD in lethally irradiated (8.5 Gy) BALB/c recipients with 5×10⁶ T lymphocyte-depleted bone marrow and 1×10⁶ splenic T lymphocytes from C57Bl/6 (allo) donors.

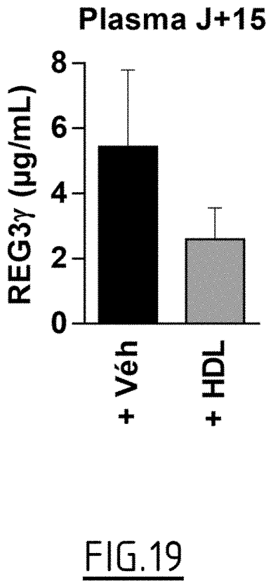

The inventors showed that IV HDL administration appears to limit the plasma level of REG-3γ, the intestinal biomarker of acute GvHD, d+15 post transplantation (n=8 mice/group in 1 experiment) (FIG. 19).

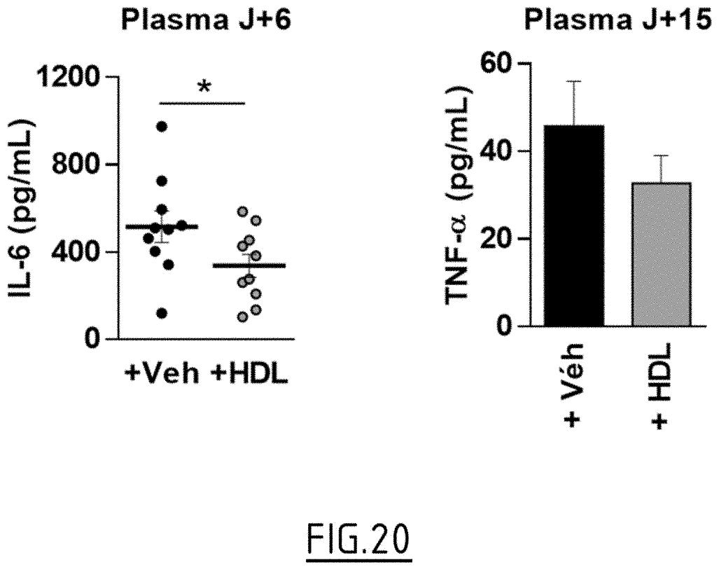

The inventors also showed that the inflammatory cytokine levels tended to be decreased by IV HDL administration: interleukin-6 (IL-6) was twice as low at d+6 (n=10 mice/group in 2 independent experiments, unpaired t test, *: p<0.05) (FIG. 20, left), and tumour necrosis factor-α (TNF-α) was decreased by 30% at d+15 (n=3 mice/group in 1 experiment) (FIG. 20, right).

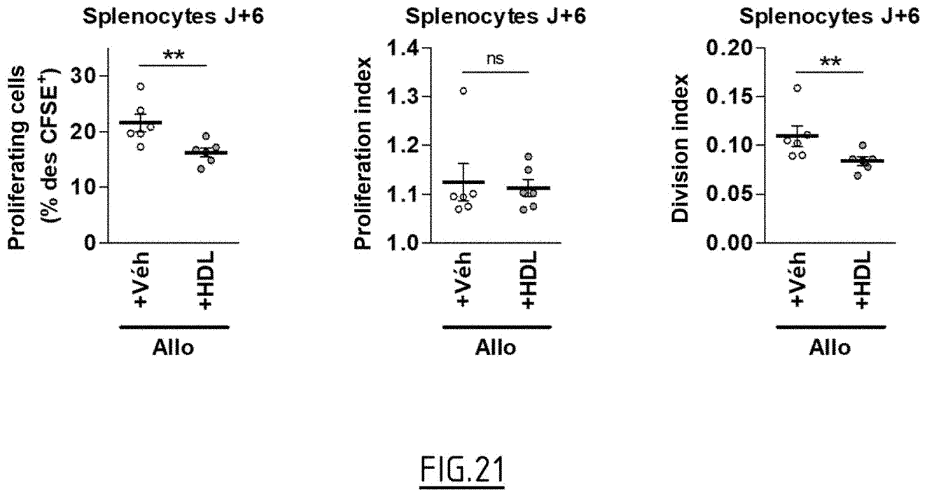

Lastly, the inventors showed that splenocytes from allotransplanted mice treated by IV HDL administration were less able to stimulate the proliferation of allogeneic naïve lymphocytes. In brief, splenocytes from mice receiving the C57Bl/6→BALB/c model were treated with mitomycin C and cultured with T lymphocytes from naïve, CFSE-marked C57Bl/6 mice. The dilution of CFSE marking was analysed by flow cytometry after 5 days of coculture. The proportion of proliferated T lymphocytes is significantly lower when they are cultured with splenocytes isolated from mice treated by IV HDL administration (FIG. 21, left). This lower proportion appears to be due to a lower number of cells dividing (division index) (FIG. 21, left) and not to a difference in division speed (proliferation index) (FIG. 21, centre) (n=6 mice/group in 1 experiment, Mann-Whitney test, ns: insignificant, **: p<0.01).

### Example 9

In this example, the inventors will show preliminary data showing that IV HDL administration limits the production of the proinflammatory cytokines TNF-α and IL-12 by hepatic macrophages, and the infiltration of CD8 T lymphocytes producing IFN-γ in the liver. This is reflected in a reduced hepatic GvHD histology score in mice treated by IV HDL administration, and more particularly in less cholangitis (i.d. inflammation of biliary tracts).

Preliminary data obtained by the inventors suggest that IV administration of HDL isolated from human plasma (20 mg/kg) 3 times per week limits hepatic GvHD in lethally irradiated (8.5 Gy) BALB/c recipients with 5×10⁶ T lymphocyte-depleted bone marrow and 1×10⁶ splenic T lymphocytes from C57Bl/6 (allo) donors.

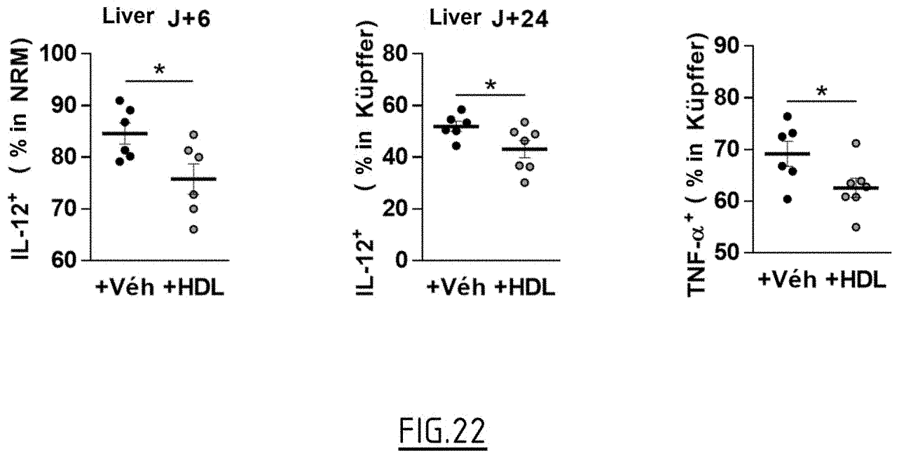

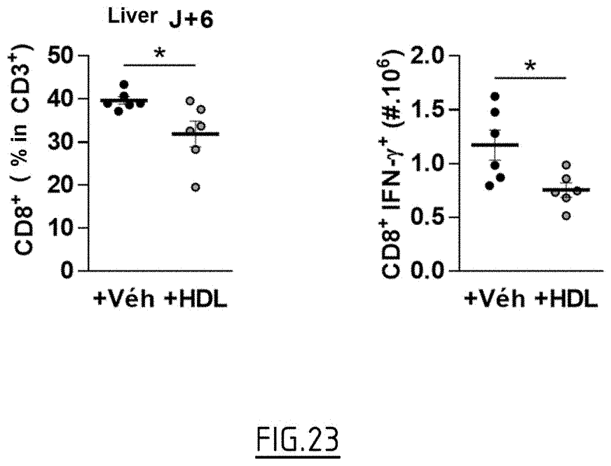

Cytokine production by non-resident (NRM) (FIG. 22, left) and resident (Küpffer cells) hepatic macrophages (FIG. 22, centre and right), and CD8 T lymphocytes (FIG. 23) was elevated, by flow cytometry 6 or 24 d post transplantation in lethally irradiated (8.5 Gy) BALB/c mice implanted with 5×10⁶ T lymphocyte-depleted bone marrow and 1×10⁶ splenic T lymphocytes from C57Bl/6 (allo) donors injected with isotonic saline (+Veh) or HDL (+HDL).

The inventors showed that the percentage of non-resident hepatic macropages (NRM) expressing IL-12 was reduced in HDL recipients (+HDL) 6 d post transplantation and after 4 h of in vitro LPS stimulation (FIG. 22, left) (n=6 mice/group in 1 experiment, Mann-Whitney test or unpaired t test, *: p<0.05). The percentages of resident hepatic macrophages (Küpffer cells) expressing TNF-α (FIG. 22, left) or IL-12 (FIG. 22, centre) were reduced in HDL recipients (+HDL) 24 d post transplantation et after 4 h of in vitro LPS stimulation (n=6 mice/group in 1 experiment, Mann-Whitney test or unpaired test, *: p<0.05).

The inventors also showed that the percentage of CD8 T lymphocytes in the liver of HDL recipient mice (+HDL) was reduced 6 d post transplantation (FIG. 23, left) (n=6 mice/group in 1 experiment, Mann-Whitney test, *: p<0.05). These CD8 T lymphocytes infiltrating the liver synthesised less IFN-γ in HDL recipient mice (+HDL) after 4 h of phorbol-myristate-acetate/ionomycin stimulation. The absolute number of CD3⁺ CD8⁺ cells expressing IFN-γ (Tc1 lymphocytes) was less elevated in mice receiving HDL injections (+HDL) than those receiving isotonic saline (+Veh) (FIG. 23, right) (n=6 mice/group in 1 experiment, Mann-Whitney test, *: p<0.05).

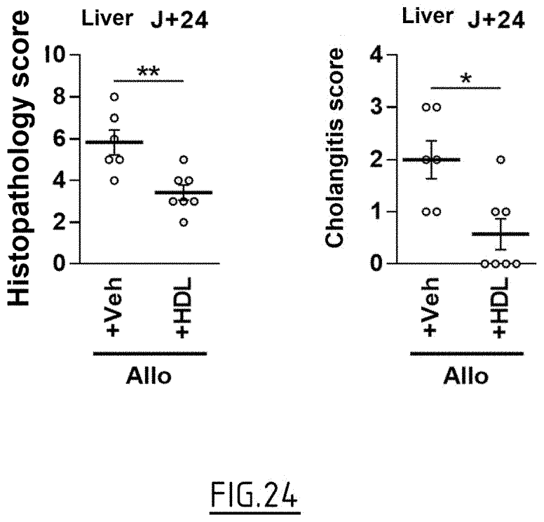

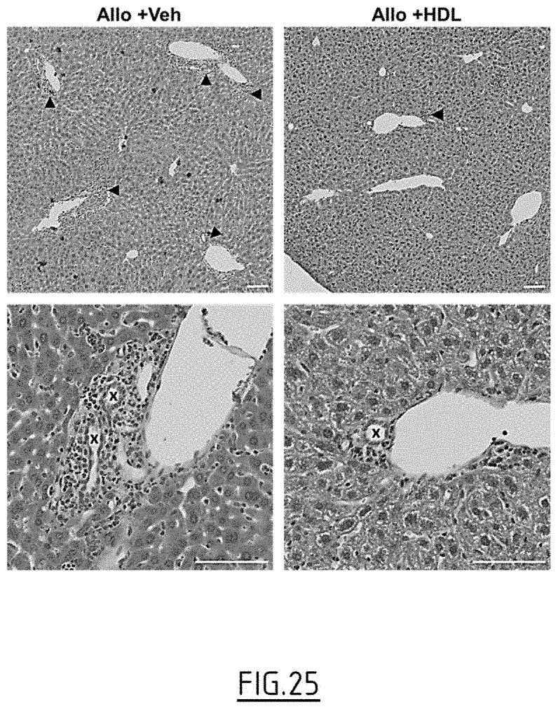

Lastly, the inventors showed that the histological score (FIG. 4) of thin liver slices fixated in formalin, then embedded in paraffin and haematoxylin/eosin stained, analysed 24 d post transplantation, taking into account the following 4 criteria: lobular hepatitis, inflammatory portal infiltrate (as shown in FIG. 25, top, arrows indicate immune infiltrate in the portal areas, scale: 100 μm), portal vein endothiliitis, and cholangitis (as shown in FIG. 25, bottom; crosses indicate bile ducts, scale: 100 μm) (each parameter evaluated on a scale of 0-3, with 0 being physiological condition)—was reduced in mice treated by HDL administration (+HDL) compared to the scores of mice receiving isotonic saline (+Veh) (FIG. 24, left) (n=6-7 mice/group in 1 experiment, test Mann-Whitney, *: p<0.05, **: p<0.01). The main effect of IV HDL administration was a reduction in cholangitis, i.e. bile duct inflammation (FIG. 24, right).

1. A method for prevention and/or treatment of graft-versus-host disease (GvHD) or cytokine release syndrome, comprising administering a therapeutically effective amount of an High-density lipoprotein (HDL) or HDL mimetic in a subject in need thereof.

2. The method according to claim 1, wherein the HDL is isolated from the blood of a healthy donor.

3. The method according to claim 1, wherein the HDL or HDL mimetic is administered intravenously.

4. The method according to claim 1, wherein the HDL (mimetic) is administered repeatedly.

5. The method according to claim 1, wherein the GvHD is acute GvHD.

6. The method according to claim 1 for the prevention of GvHD, wherein the subject is to undergo a haematopoietic cell transplant.

7. The method according to claim 6, wherein the HDL (mimetic) is administered before and after the haematopoietic cell transplant.

8. The method according to claim 1 for the treatment of GvHD, wherein the subject has undergone a haematopoietic cell transplant.

9. The method according to claim 1, for the prevention and/or treatment of cytokine release syndrome, wherein the subject is treated with chimeric antigen receptor T lymphocytes (CAR-T).

10. The method according to claim 4, wherein the HDL (mimetic) is administered every 2 days.
